(19) United States
(12) Patent Application Publication
(10) Pub. No.: US 2010/0016662 A1
(43) Pub. Date: Jan. 21, 2010

(54) Radial Scanner Imaging System

(75) Inventors: Kenneth Edward Salsman, Pleasanton, CA (US); Anders Grunnet-Jepsen, San Jose, CA (US); Brian Glenn Jamieson, Severna Park, MD (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Innurvation, Inc.

(21) Appl. No.: 12/389,202

(22) Filed: Feb. 19, 2009

Related U.S. Application Data
(60) Provisional application No. 61/030,453, filed on Feb. 21, 2008.

(51) Int. Cl. A61B 1/04; H04N 7/18

(52) U.S. Cl. 600/109; 348/68; 348/E07.085

(57) ABSTRACT

A radial scanner configured to image the interior of a tube includes, in an embodiment, a housing having a transparent window, a photo-sensing array, a mirror located within the housing and oriented to direct an image around a circumference of an interior surface of the tube outside the transparent window to the photo-sensing array, and a light source configured to illuminate the interior surface of the tube, wherein the photo-sensing array is configured to receive the image around the circumference as a circular line scan. In an embodiment, the radial scanner is deployed as an ingestible capsule. In another embodiment, the radial scanner is deployed as a fiber optic catheter.

### CROSS-REFERENCE TO RELATED APPLICATIONS

The present application claims the benefit of U.S. Provisional Patent Appl. No. 61/030,453, filed Feb. 21, 2008, which is incorporated herein by reference in its entirety.

### BACKGROUND

1. Field

Embodiments of the present invention relate to imaging systems, in particular imaging systems for use inside tubular structures of the body, such as intestines and veins.

2. Related Art

Borescopes, endoscopes, and similar tools used to inspect or image tubular structures have relied on classic camera optical system designs, where a lens is used to capture a full 2D image that is then focused down an optical fiber or projected onto an electronic image capture device such as a CCD or CMOS imaging focal plane array (FPA). This requires the optical system to have a very wide angle lens so as to capture the image of the tubular wall closest to the optics, or to provide a flexible or mirror deflective system so that the operator can move the field of view into the realm of 90 degrees from the axis of the tubular structure being examined. As a result of this design approach, several performance issues occur. A conventional camera having these performance issues is illustrated in FIG. 18.

One performance issue is distortion. Systems using wide-angle lenses suffer from “fisheye” effects in the image where objects lose their relative position and size depending on their location within the image. Likewise, in tubular environments, the highest resolution can be achieved while imaging surfaces that are closest to the lens. Wide angle lenses provide the ability to see the circumference of the tubular structure, but suffer in image quality as the light from the surface undergoes more radical deflection. This means that a lens that is looking down a tube with the center of the tube and the center of the lens in alignment will provide an image of the closest surfaces in the region of the lens where there is more optical distortion. Pixels in this peripheral region of the lens typically show significant coma or comet-like shapes, and lose their true geometric format. In a performance paradox, the center of the field of view where there is the least optical distortion is where the angle of the surface and the distance to the surface is the worst, and provides the least useful image performance.

A second performance issue is variable resolution. A camera looking down the axis of a tube will see the highest resolution of the surface at the closest point of focus to the lens. However, the angle of the surface to the axis of the camera causes the area seen by any one pixel in the imaging system to increase rapidly as the location of the surface being imaged moves away from this closest point. This steep angular orientation produces a drop in resolution and a distorted perspective of the surface features that are visible.

Another performance issue is variable illumination. As the tubular surface being examined by a co-axial camera moves further from the camera, the steep angle to the surface further decreases the amount of effective illumination of the surface. This causes a rapid drop in relative brightness and produces a highly non-linear illumination within the field of view. To maximize the amount of surface being viewed in each image, the closest surfaces are typically overexposed, further reducing the quality of the image.

Yet another performance issue is loss of useful pixels. Cameras use either film or focal plane arrays designed to capture images in a two-dimensional planar manner. As a result, images taken down the axis of a tubular structure are akin to the proverbial round peg in a square hole. The image of a tube taken with a wide angle lens that is in axial alignment with the tube shows a circular image area where the region between the circular area and the rectangular area of the photo sensing imager remains unused. This typically results in a loss of about 20% of the resolution. Further, due to the extreme angle to the surface and resultant poor lighting, the surface area in the center of the field of view is of exceedingly low value. This leaves only a ring of pixels in each image where the pixel value is high enough to provide the user with viable information.

Another performance issue is the requirement for dynamic range. The geometry issues with axial imaging of a tubular structure involve the variance in intensity of illumination from the distant surface in the center of the field of view to the nearby surface area at the periphery of the field of view. This generates a very wide range of illumination intensities, which increases the difficulty in producing an image with a maximum of valuable data. When the mid-range tissue is illuminated to the correct level for maximum contrast, the tissue near the camera will be overexposed, and the tissue in the center of the field of view will be underexposed. This situation limits the ability of the system to collect optimized images of the entire visible surface region. As such, systems taking images at a time interval while moving through the tubular structure produce data sets where significant regions are either not in the image or are not imaged at an optimal illumination level.

Obtaining the required depth of focus is also difficult. Due to the long range of distances within the field of view of an axially oriented camera, the system must either take multiple images at various focus settings, utilize a high stop setting (that is, minimize the aperture of the lens) or use a lens which has a long focal length relative to its diameter. These options all allow the ability to generate a final image with a majority of the image in focus. However, there are significant trade-offs in these options with very negative impacts. Use of multiple exposures at multiple focus settings can allow for a composite image to be generated with essentially the entire tubular surface in focus. This technique is used in microphotography of biological tissues to allow vascular and cellular structures with large, three-dimensional structures to be captured fully in focus. The capture of multiple images takes significant time and power, and generates very large data files for imaging a single region of the surface. Use of an aperture stop or long focus optical system both result in large depths of focus, but they also are very inefficient in utilization of light. Long focal length lenses also do not have the ability to focus on nearby surfaces, thereby limiting the ability of this approach to produce good images of the region where a wide angle lens provides its best performance and the surface is at the best location for imaging.

Another performance issue is an inability to provide an appropriate F-stop for the entire image. As mentioned previously, optical systems may be equipped with an optical stop or restriction which reduces the relative aperture of the lens. This results in an increase of the focal range, but at the expense of the optical efficiency of the system. By reducing the functional size of the lens, the light collection ability of the optics is reduced, resulting in the need for higher levels of illumination and more power needed to drive the imaging system. This has a very detrimental impact on any system being designed for portable or battery operation.

Steerable or restricted field of view systems also suffer from performance issues. Endoscopes are typically built with flexible fiber-optic heads that allow the user to bend the end of the scope and look at the sidewall of the tubular structure. Likewise, bore scopes can be equipped with 90 degree turning mirrors so that the unit can be used to view the side wall of the tubular structure it is inserted into. When these units are operated in this manner, they lose the ability to see the entire circumference of the tube. This restriction of the field of view requires the operator to keep track of parts of the wall surface that have been viewed. It also significantly increases the amount of images and time required to view the surface.

### BRIEF SUMMARY

A radial scanner may be used to image the interior of a tube, such as an intestine or vasculature of an animal. Radial scanners can include optical systems capable of utilizing one or more photo sensors and a mechanical or electro-mechanical scanning optical element. This creates a line scan or a solid state array of photo sensors arranged in a format such that light from the subject can pass through the optics, which collects it in a circular format and onto the sensor array. In an embodiment, these optical elements include a standard camera imager and a lens axially aligned to a cylindrically-symmetrical mirror such as a cone mirror, which directs the light from the subject at a 90 degree deflection from the target surface to the lens. The lens focuses the image onto a ring of photo sensitive pixels fabricated on a semiconductor substrate.

In an embodiment, a ring array of prisms, lenses, and/or multi-faceted or conical mirrors may be used which capture the light from the target surface and direct it in at an angle from incidence to a second optical element. The second optical element includes image-forming elements which direct the light onto a pattern of photo sensitive pixels to produce an electronic image of the scan region. These multi-element optical arrays can be fabricated from single material structures, providing the ability to keep them in position. By utilizing alignment pins and spacers, the optical system may be easily assembled and aligned, with each element in the array kept at its appropriate location for optimal and uniform performance.

In another embodiment, fiber optical elements may be used to collect the light from the target surface through a lens element in the tip. Each element may then be directed to a pixel array to provide an image of the radial scan region.

Further embodiments, features, and advantages of the present invention, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is an illustration of a radial scanner system according to an embodiment of the present invention.

FIG. 2 is an exemplary image having distortion caused by a cone-shaped optical element.

FIG. 3 is an exemplary result of processing performed on the image of FIG. 2.

FIG. 4 is an illustration of a radial scanner system according to another embodiment of the present invention.

FIG. 5 is an exemplary image of text.

FIG. 6 is an exemplary distorted image obtained according to an embodiment of the present invention.

FIG. 7 is an exemplary image with distortion removed according to an embodiment of the present invention.

FIG. 8 is an illustration of a radial scanner system having an illuminator according to an embodiment of the present invention.

FIG. 9 is an illustration of a radial scanner system having an illuminator according to another embodiment of the present invention.

FIG. 10 is an illustration of a radial scanner system according to another embodiment of the present invention.

FIG. 11 is an illustration of a radial scanner system according to another embodiment of the present invention.

FIG. 12 illustrates an ingestible capsule according to an embodiment of the present invention.

FIG. 13 illustrates an ingestible capsule according to another embodiment of the present invention.

FIG. 14 illustrates a tethered device according to an embodiment of the present invention.

FIG. 15 illustrates a combination camera and radial scanner system according to an embodiment of the present invention.

FIG. 16 is a flowchart of a method for obtaining a high-resolution image according to an embodiment of the present invention.

FIG. 17 illustrates an exemplary image as viewed by a radial scanning system according to an embodiment of the present invention.

FIG. 18 illustrates a conventional axial camera.

FIG. 19 illustrates an exemplary reflective element according to an embodiment of the present invention.

FIG. 20 illustrates an exemplary reflective and refractive element according to an embodiment of the present invention.

The present invention will be described with reference to the accompanying drawings. The drawing in which an element first appears is typically indicated by the leftmost digit(s) in the corresponding reference number.

### DETAILED DESCRIPTION

While specific configurations and arrangements are discussed, it should be understood that this is done for illustrative purposes only. A person skilled in the pertinent art will recognize that other configurations and arrangements can be used without departing from the spirit and scope of the present invention. It will be apparent to a person skilled in the pertinent art that this invention can also be employed in a variety of other applications.

It is noted that references in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described.

A side-scanning optical system can provide improvements over conventional axial camera systems when used in tubular environments. In particular, scans of the interior surface of a tube, such as an intestine, vasculature, urinary tract, reproductive tract, lung, nasal or internal ear cavity of an animal, can provide high-resolution images of the interior of the tube in an easily-examined format without a need for a surgical extraction (e.g., biopsy) of the tissue. For instance, directly scanning the surface of an intestine rather than viewing the intestine down the intestinal path allows a doctor to examine the intestine as if it has been cut apart and laid flat as a familiar viewpoint but without the need of a biopsy under microscope, rather than current state of the art requiring the doctor to determine surface features from a non-optimal viewpoint.

Embodiments of the present invention afford a doctor or medical professional to be able to view a single static display of the image of a full surface scan of a tubular structure or cavity. Current state of the art displays are in a bouncy, confusing format of a series of still shots or combined into a movie presentation, but do not show the entire length of a tube at any single moment. This forces a reviewer to spend time transiting the images and/or movie. Embodiments of the present invention allow a reviewer of a full scan to very rapidly diagnose a condition, providing the medical diagnostic industry both the savings of large amounts of time and money, but also provides an ability to increase a diagnostic effectiveness by spending quality time in the detail of an area of interest.

Flat bed or linear scanners utilize a line of photo sensors that are arranged in one or more rows, depending on how the system detects color. These rows of photo sensors capture images of flat objects that are either moved across the detector array in a perpendicular direction to its orientation or where the array is moved over the surface to be imaged in a similar directional manner. Such flat objects may include papers, books, photos, etc. However, these types of linear scanners are not conducive to imaging non-linear objects, such as the interior of a tube.

As will be described herein, a radial scanner may be used to image the interior of a tube. Radial scanners can include optical systems capable of utilizing one or more photosensors and a mechanical, electro-mechanical, or solid state scanning optical element. This creates a line scan or a solid state array of photosensors arranged in a format such that light from the target surface can pass through the optics, which collects it in a circular format and directs it onto the sensor array.

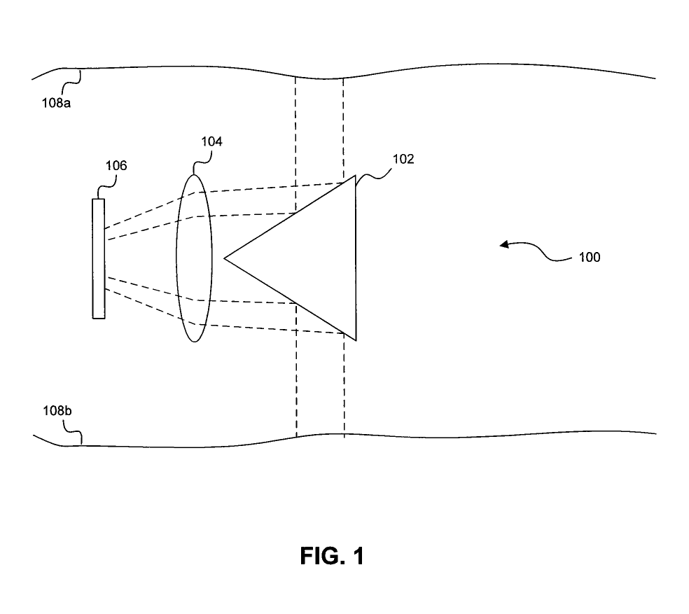

FIG. 1 is an illustration of a radial scanner system 100 according to an embodiment of the present invention. Radial scanner system 100 includes a cone-shaped mirror 102, a lens 104, and a sensor 106.

Radial scanner system 100 may be used to image the interior of a tubular structure 108. In a body, tubular structure 108 may be, for example and without limitation, part of the digestive system, such as an intestine, or part of the vascular system, such as a vein or artery. Tubular structure 108 is illustrated in FIG. 1 as a cross-section such that interior surfaces 108a and 108b are imaged by radial scanner system 100. One of skill in the art will recognize that in practice, radial scanner system 100 may image completely or partially around the interior circumference of tubular structure 108.

An image from surfaces 108a, b is reflected from cone-shaped mirror 102 towards lens 104. In an embodiment, the angle of reflection is approximately 90 degrees. Lens 104 may be, for example, a standard camera lens axially aligned to cone-mirror 102 such that light reflected from cone-shaped mirror 102 is directed onto sensor 106. Sensor 106 may be, for example, a light-detecting array, such as photosensitive pixels fabricated on a complementary metal oxide semiconductor (CMOS) substrate.

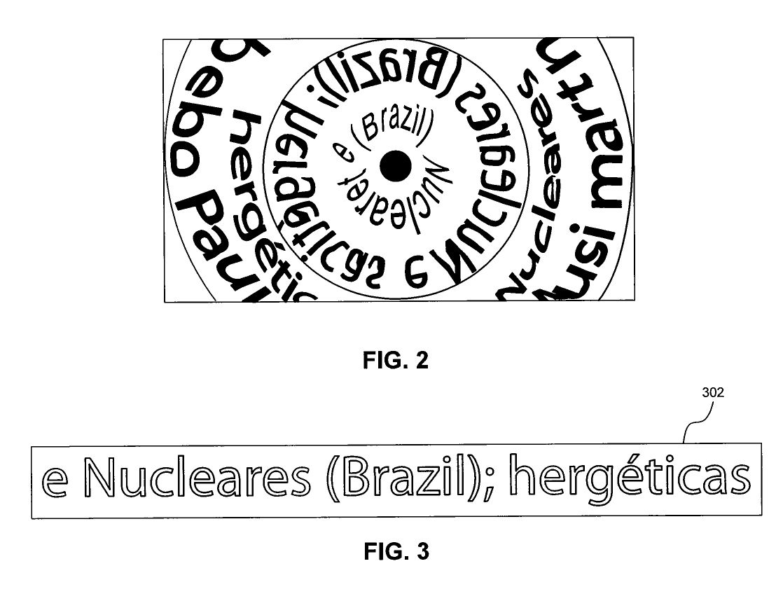

Lens 104 focuses and/or directs the image to sensor 106. Because mirror 102 is cone-shaped, the image received by sensor 106 is circular in nature. FIG. 2 illustrates an example image 202 having a circular image similar to that caused by cone-shaped mirror 102. This circularity may be removed in a post-processing software system, such that the image is uncurled into a linear dimension. FIG. 3 is an example result of processing performed on circular image 202 to create a linear image 302.

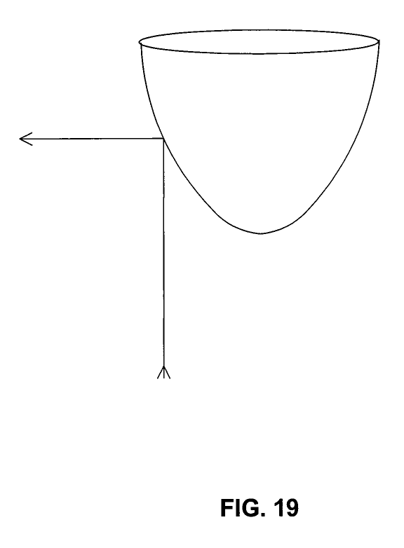

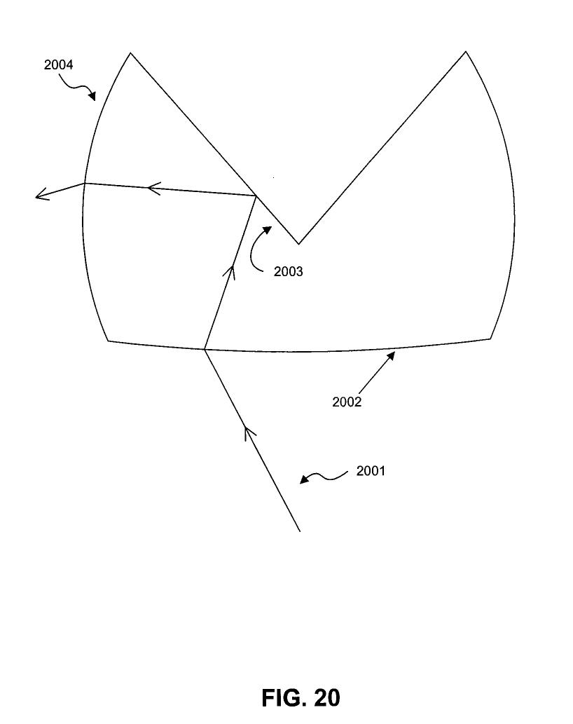

In this description, it will be obvious to one skilled in the relevant art that the cone-shaped reflective element is described for illustrative purposes only. In other embodiments, another cylindrically-symmetrical shape such as a parabola or other solid of rotation could be utilized to reflect light from the strip of imaged tissue onto the focal plane array (FIG. 19). In some embodiments, the reflective element could in fact be made of multiple surfaces, some of which are refractive and some of which are reflective. In FIG. 20, for example, a ray of light 2001 is first refracted (focused) at surface 2002, and then reflected from the silvered surface 2003, and finally reflected (focused) again at surface 2004. In this way, a single cylindrically-symmetrical optical element can contain both corrective (focusing) elements and the required orthogonally-reflective, cylindrically-symmetrical element needed for 360 degree scanning optics as described herein.

In a typical camera system, a sensor array may have, for example, 320 columns×240 rows of pixels, resulting in a total pixel count of 76,800 pixels.

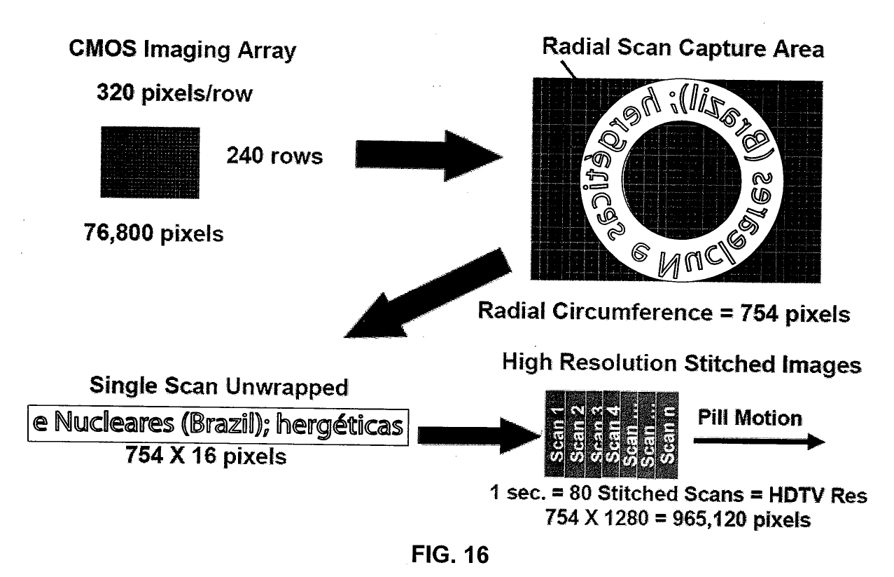

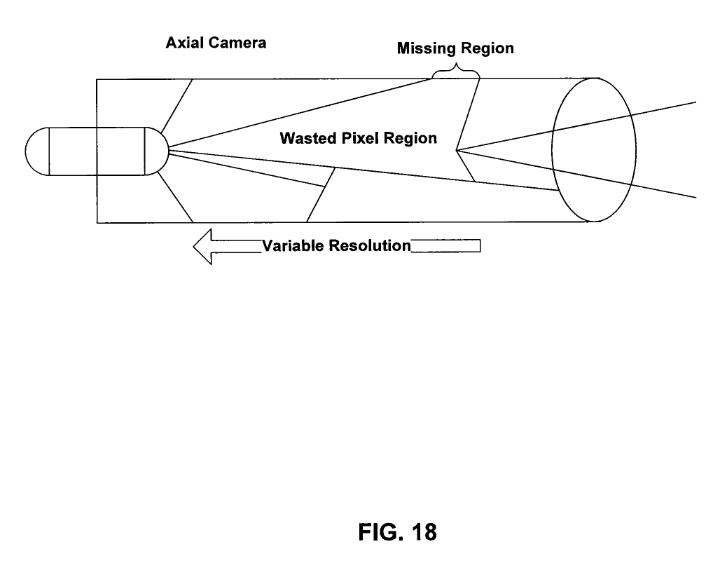

In a radial scanner system, however, the image is wrapped around the circumference of a circle. If the same size array used in a typical camera is used in a radial scanner according to an embodiment of the present invention, the effective pixel count for a single layer of pixels around the circumference of the image is approximately 754 pixels. If each image has a circular line depth of, for example, 16 pixels (e.g., the number of circles of pixels in the image is 16), the effective total pixel count is approximately 754×16 pixels, or approximately 12,000 pixels per scan. Because the effective pixel count within a line (754 as opposed to 320) is higher than a typical camera system, the line resolution of the radial scanner system is also higher than in a typical camera system utilizing the same image sensor. Obtaining this high resolution image is further described in FIG. 16.

When imaging the interior of a tubular structure, the surface of the structure closest to the imager is of most interest to the viewer. For example, when imaging the interior of the intestines, the lining of the intestine, rather than the view down the path of the intestine, is of most interest when the viewer is searching for specific features on the intestinal wall. Additionally, the area of the image near the edge of the field of view is of higher resolution than the center portion of the field of view, since the pixel count is higher with greater image circumference. Therefore, a radial scanner system such as system 100 provides the highest resolution in the area of the image of most interest to a viewer.

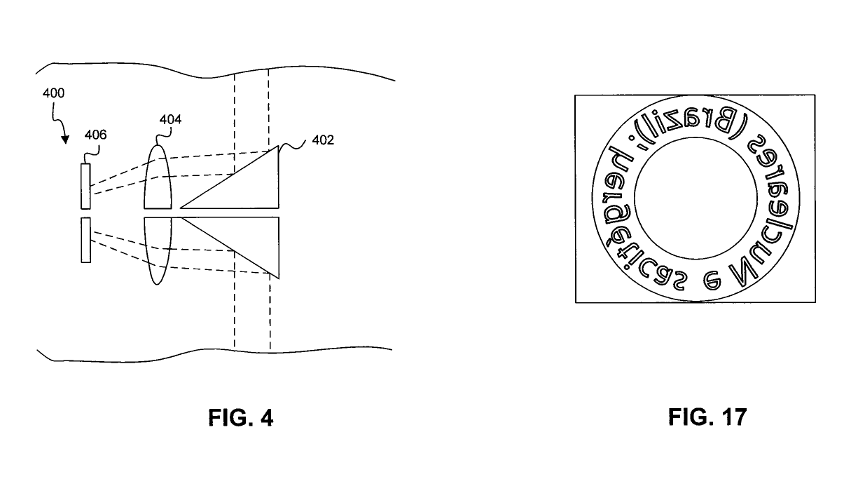

FIG. 4 is a cross-section of a radial scanning system 400 that takes advantage of this feature to provide a system requiring less power than system 100. Since the center of the field of view provided by the cone-shaped mirror (that is, the view down the tube) is of minimal interest, cone-shaped mirror 402, lens 404, and sensor 406 may be hollowed out so that only light from the edge of the field of view is imaged by sensor 406. Because sensor 406 only needs to power a ring of subsensors as opposed to a full rectangular array of subsensors, the power consumed by system 400 is reduced as compared to system 100. In an embodiment, the hollowed-out portion of the system is used for structural supports, such as spacers, as well as a pass-through for wiring or other necessary supports such as a method for illumination. FIG. 17 illustrates an unprocessed image as viewed by radial scanning system 400.

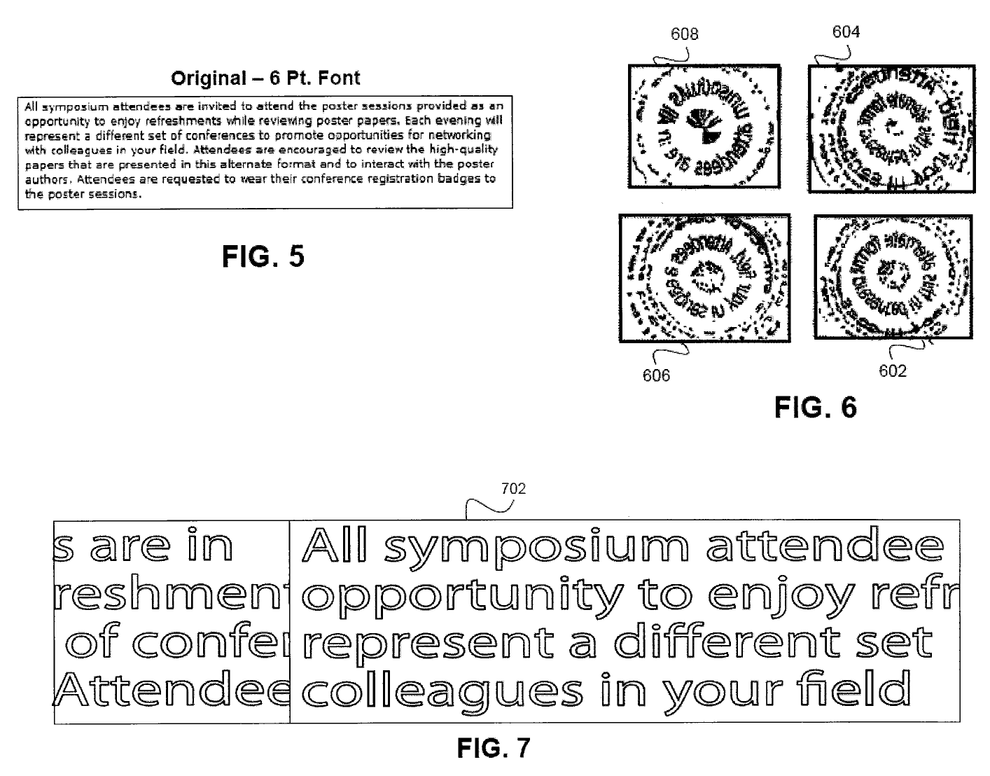

Returning to the system of FIG. 1, as radial scanner system 100 proceeds through a tube, consecutive images may be captured. Each of the consecutive images, once circularity is removed, may then be stitched together to form a full scan of the interior of the tube. FIGS. 5-7 illustrate such a process. FIG. 5 is a sample of text to be imaged. In FIG. 6, the sample of text from FIG. 5 has been coiled into a tube. Images 602, 604, 606, and 608 are resultant images from passing a radial scanning system similar to system 100 through the tube. Each of images 602, 604, 606, and 608 are taken at different locations within the tube. FIG. 7 illustrates the post-processing result of images 602, 604, 606, and 608. Each image has been processed so as to remove the circularity and produce a linear image. The multiple linear images are then stitched together to produce a scanned image 702.

The surface to be imaged may be illuminated by a light source associated with system 100. For instance, illumination may be provided by one or more light emitting diodes (LEDs). Although the illumination source will be described as including LEDs, one of skill in the art will recognize that other types of light sources, whether pulsed or constant, may also be used without departing from the spirit and scope of the present invention.

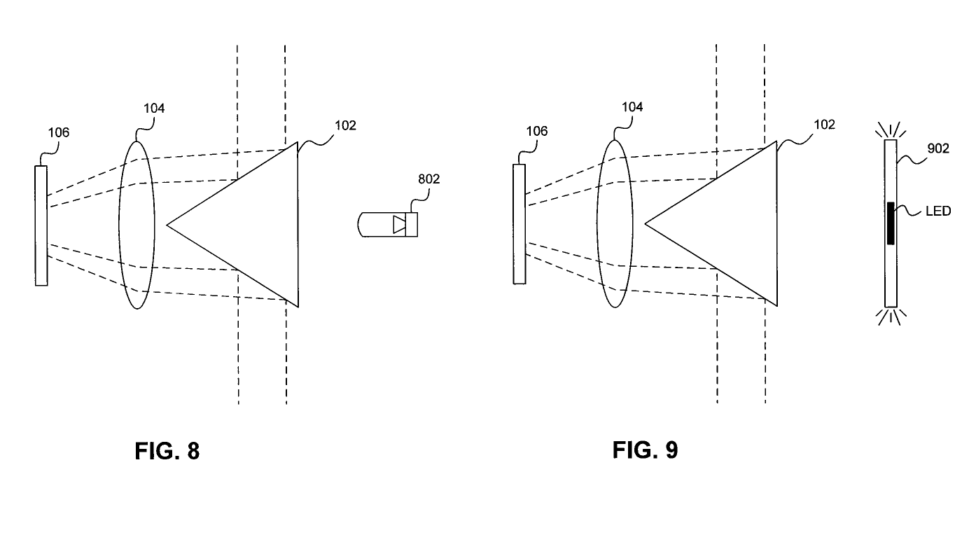

In an embodiment, one or more LEDs may be located around the edge of sensor 106. In another embodiment, one or more LEDs may be located on the side of cone-shaped mirror 102 opposite sensor 106. FIG. 8 illustrates such an embodiment. As shown in FIG. 8, positioning light source 802 behind cone-shaped mirror 102 reduces the possibility of direct light source light reaching sensor 106. In an embodiment, one or more LEDs may be placed inside an optical light dispersing ring, such that light is emitted from the illuminator in a radial strip corresponding to a section of a surface to be imaged. FIG. 9 illustrates such an optical ring 902.

If multiple light sources are used, the light sources may be of varying colors so that spectral selectivity within an image is possible. In an embodiment, red, green, and blue LEDs are used to illuminate the target surface. Sensor 106 may have different photosensing elements corresponding to the different colors so that the image is separable into spectral channels. In embodiments, very narrow bands of frequencies may be used within the general band spanned from red through blue. In additional embodiments, however, extending the spectrum into infrared and ultraviolet frequencies may provide other diagnostic data. One of skill in the art will recognize that a specific selection of light frequencies corresponding to a specific diagnostic test may differentiate several products in a family of diagnostic products based on the disclosure herein.

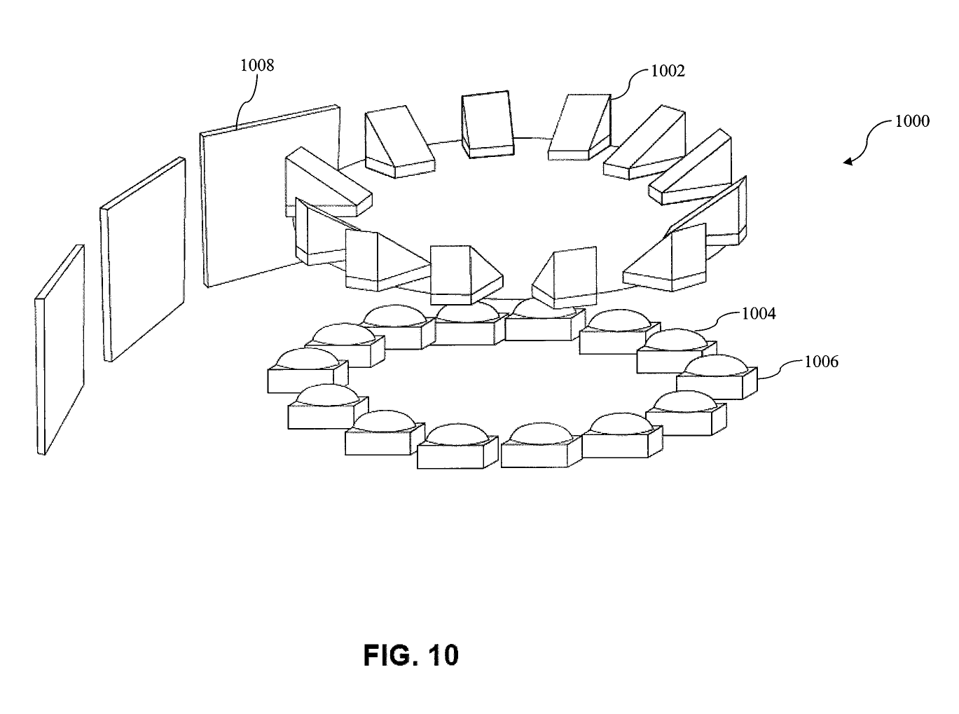

FIG. 10 illustrates a radial scanning system 1000 according to another embodiment of the present invention. Radial scanning system 1000 includes a radial set of folding mirrors or prisms 1002, a radial set of microlenses 1004, and a radial set of photosensors 1006. An image from the target surface having a field of view 1008 enters a respective folding mirror 1002. Folding mirror 1002 reflects the image onto a respective microlens 1004. In the example shown in FIG. 4, folding mirrors 1002 include prisms, in which each prism has a reflective hypotenuse surface such that light is reflected through the interior of the prism. One of skill in the art will recognize that other types of folding mirrors may be used without departing from the spirit and scope of the present invention. The corresponding microlens 1004 directs the image onto a respective photosensor 1006 to produce an electronic image of the scan region. Fields of view 1008 may overlap, such that the images produced by each photosensor 1006 corresponding to respective fields of view 1008 may be stitched together to create a continuous image. Folding mirrors 1002 are provided for a convenience of locating microlenses 1004 and photosensors 1006 conveniently on a single planar surface. However, use of folding mirrors 1002 is not necessary. In an embodiment, microlenses 1004 and photosensors 1006 may be mounted upon a flexible surface that is wrapped around itself in a circular fashion. This alternate embodiment assumes a more complicated assembly and electronic routing scheme. Although this alternate embodiment is not depicted herein, the assembly of the system would be apparent to one of skill in the art based on the disclosure provided herein.

Multi-element optical arrays 1002 and 1004 can be fabricated from single material structures, providing the ability to keep them in position. For example, radial set of folding mirrors 1002 may be created or fabricated from a single piece of material. Similarly, radial set of microlenses 1004 may be created or fabricated from a single piece of material. By utilizing alignment pins and spacers, the optical system may be easily assembled and aligned, with each element in the array kept at its appropriate location for optimal and uniform performance.

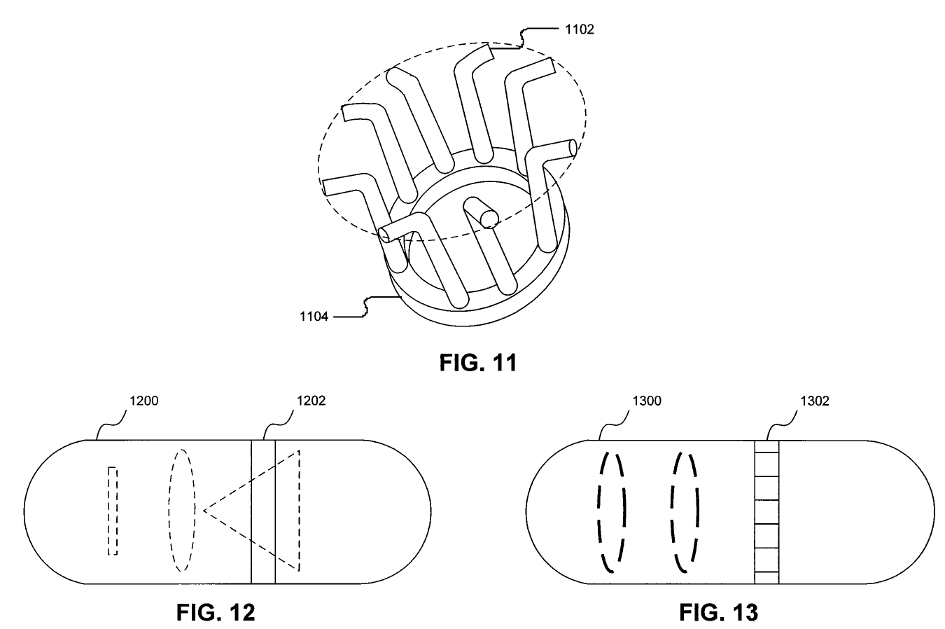

FIG. 11 illustrates a radial scanner system 1100 according to another embodiment of the present invention. Radial scanner system 1100 includes fiber optical elements 1102 connected to a ring of photosensing pixels 1104. Each of fiber optical elements 1102 may include a lens located on the tips of the fiber optical elements opposite ring of photosensing pixels 1104. In this manner, each of fiber optical elements 1102 acts as a light pipe, such that light enters through the open end of a fiber optical element 1102 and is transmitted directly to a corresponding photosensing pixel on ring 1104. Fiber optical elements 1102 may be bent at an angle needed to image an interior surface of a tube. For example, fiber optical elements 1102 may be bent such that the surface of the fiber optical element connected to ring of photosensing pixels 1104 is at approximately a 90 degree angle to the surface of the fiber optical element facing the interior of the tube. One of skill in the art will recognize that the number of fiber optical elements shown in FIG. 11 is for illustrative purposes only. Additional or fewer fiber optical elements may be used depending on one or more of: the number of photosensing pixels in ring 1104, the diameter of each fiber optical element 1102, and the amount of overlap desired between images produced by each fiber optical element. Fiber optical elements 1102 may be sized to partially or completely cover a single pixel in ring of photosensing pixels 1104. Additionally, one of skill in the art will recognize that angles less or more than 90 degrees may be used when disposing means of illumination within the same system.

Radial scanning systems according to embodiments of the invention, such as those described above, can capture multiple radial lines during any one scan to provide both color and the ability to detect and measure the motion in the axial direction perpendicular to the scan line. This ability to measure the amount of motion and direction of motion between each scan allows the scanning system to accurately stitch each scan into a continuous image of the full tubular surface. In addition, the ability to measure the motion incorporated with the known size of the pixel allows accurate measurement of the distance traveled down the tube, as well as providing accurate measurement of the size of objects observed on the tubular wall.

In one embodiment, motion is detected within an endoscopic capsule system. Motion detection within the capsule may prevent mostly or completely duplicate scans from consuming limited energy reserves without need. A motion detection device may be external to a radial scanning subsystem or included within a radial scanning subsystem.

In another embodiment, motion is detected external to an endoscopic capsule by or through a receiver worn or proximal to a patient. In this configuration, a larger (external to the body) energy reserve is available to determine motion and the extent of motion. In such an embodiment, the external system has a means for communication and control of the endoscopic capsule to deliver one or a series of scans upon the external command. In this embodiment, the endoscopic capsule can be very efficient with limited energy reserves.

Radial scanner systems such as those described above may be adapted for use in both tethered and non-tethered devices. Tethered devices include, for example and without limitation, endoscopes and medical catheters. Non-tethered devices include, for example and without limitation, an ingestible capsule or pill. Use in such devices will be described in further detail below.

FIG. 12 illustrates an ingestible capsule 1200 according to an embodiment of the present invention. Ingestible capsule 1200 contains a radial scanning system similar to that described with respect to FIG. 1. Light from surfaces around the circumference of capsule 1200 may enter the radial scanning system through aperture 1202.

FIG. 13 illustrates an ingestible capsule 1300 according to another embodiment of the present invention. Ingestible capsule 1300 contains a radial scanning system similar to that described with respect to FIG. 10. Light from surfaces around the circumference of capsule 1300 may enter the radial scanning system through an aperture 1302.

In a non-tethered device, such as capsules 1200 or 1300, images received by the photosensors may be stored on an on-board memory device and/or may be transmitted to a receiver via an on-board transmitter. An ingestible capsule having an on-board memory device and/or transmitter is further detailed in U.S. patent application Ser. No. 11/851,221, filed Sep. 6, 2007 and entitled, “Ingestible Low Power Sensor Device and System for Communicating with Same,” and U.S. patent application Ser. No. 11/851,214, filed Sep. 6, 2007 and entitled, “System and Method for Acoustic Information Exchange Involving an Ingestible Low Power Capsule,” each of which is incorporated by reference herein in its entirety. In other embodiments, images may be transmitted to a receiver via radio frequency (RF) emissions by typical wireless means utilizing traditional antenna and RF techniques. Furthermore, in yet another embodiment of an ingestible capsule, transmission of scanned images may occur through direct electrical connection to and conductive use of animal tissue as a communication pathway.

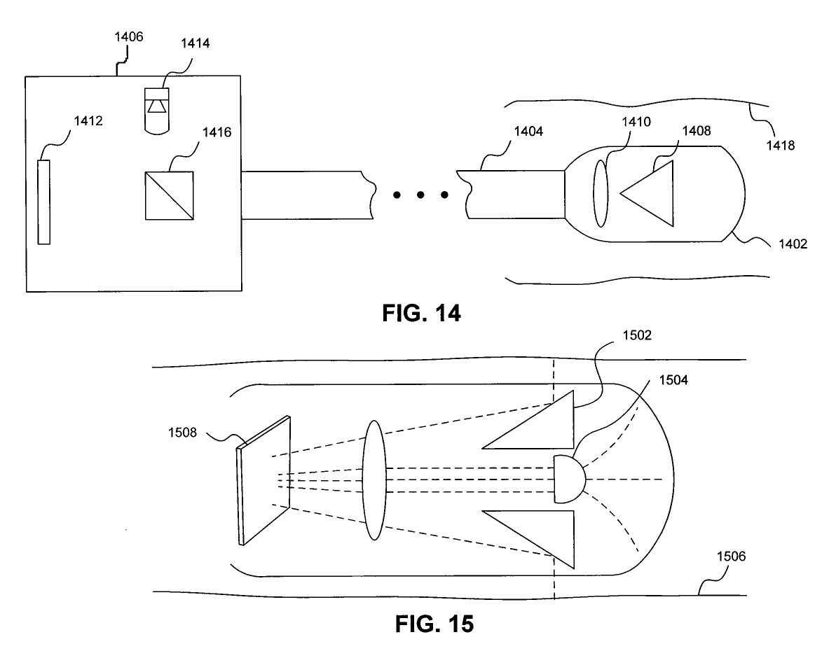

If the radial scanning system is to be used in a tethered device, components of the radial scanning system may be separated to reduce the size of an intra-cavity unit. For example, if the radial scanning system is to be used in a medical catheter, any or all of the photosensor array and the illumination source may be located external to a patient and connected to the reflector component via one or more fiber optic cables. FIG. 14 is an illustration of a tethered device 1400 according to an embodiment of the present invention. Tethered device 1400 includes an intra-cavity unit 1402 coupled to a distal end of a fiber optic cable 1404, and an extra-cavity unit 1406 coupled to a proximal end of fiber optic cable 1404. In the embodiment of FIG. 14, intra-cavity unit 1402 includes a reflector 1408 and one or more lenses 1410. Reflector 1408 may be, for example and without limitation, a cone-shaped mirror or a set of folding mirrors or prisms. In the embodiment of FIG. 14, extra-cavity unit 1406 includes a photosensor 1412, an illuminator 1414, and a beamsplitter 1416. Illumination light is transmitted from illuminator 1414 through beamsplitter 1416 and fiber optic cable 1404. The illumination light is reflected by reflector 1408 so as to illuminate a tubular surface 1418. A resulting image of tubular surface 1418 is reflected by reflector 1408 through lens 1410 and fiber optic cable 1404. The image then passes through beamsplitter 1416 and is detected by photosensor 1412. Since the power source for the radial scanning system may also be located external to the patient, a tethered device is not subject to the same power restrictions as a non-tethered device.

For example, a standard imager device, rectangular in nature, may be deployed as photosensor 1412. Use of a standard device may require inefficient use of power to collect then discard a portion of pixels not utilized. However, use of a standard imager device affords an efficiency of cost and an abundance of parts. Extra power is reasonably available in numerous methods external to a body cavity. Additionally, a tethered radial scanning device need not be concerned about limiting a rate of capturing a scan to be power efficient. Duplicate image information and the energy necessary to acquire the information can simply be discarded after post-processing.

FIG. 15 illustrates a cross-section of a combination camera and radial scanning system 1500 according to an embodiment of the present invention. As discussed above, a radial scanning system may be hollowed-out without losing significant image quality or resolution. In such an embodiment, a conventional camera may be placed in the hollowed-out portion of the radial scanning system. As illustrated in FIG. 15, combination system 1500 includes a hollowed-out cone-shaped mirror 1502 and a lens 1504. An image of tubular surface 1506 is transmitted to a photosensor 1508 as described above. An image from lens 1504, which represents the view down the tube, is transmitted to photosensor 1508 through the hollowed-out portion of cone-shaped mirror 1502. One of skill in the art will recognize that mirror 1502 and lens 1504 can be created or fabricated from a single piece of material, such as an optical quality injection molded plastic.

A combination camera and radial scanning system, such as combination system 1500, provides a user with a high-resolution image of the tubular surface as provided by the radial scanning system, as well as the context of tubular location as provided by the conventional camera. In the case of an endoscope, technicians navigate the insertion and extraction of an endoscope utilizing the view down the intestines. Combination of the camera with the radial scanner eliminates the need to turn and/or reposition the endoscope in order to view details on the side wall of the intestines.

### Advantages

Radial scanning solves many, if not all, of the performance issues associated with conventional bore scopes, endoscopes, endoscopic capsules, and the like. In a radial scanning approach, an image of the tubular structure is taken with a system that is physically oriented to travel along the axis of the tube, but whose optical system is designed to image a narrow cylindrical region of the tube's inner wall. In this manner, a series of lines capturing the circumference of the tube are captured as the scanner is moved physically down the axis of the tube. These line captures can be composed of a single pixel ring or multiple rings, such that individual scans can capture narrow bands of the wall. This allows for both capture of color images and tracking of the motion of the unit between scan captures so that the bands can be digitally overlapped to generate a seamless image of the entire length of the tubular structure. The resulting data file can then be an image that is formatted to be the circumference of the tube in one axis (taken at the pixel resolution of the scanner) and as many pixels in length as the number of pixels over the length of the tube that was scanned. Since the scanner takes its scans at 90 degrees to its axis, the resolution is uniform and constant over the region that is scanned, much like the same performance in flatbed scanners.

In an embodiment, a capsule endoscope configured with a radial scanner produces images that are processed and overlapped on a device worn by the patient or in close proximity to the patient. This embodiment affords a real time view as an endoscopic capsule is propelled by peristalsis through a gastro-intestinal tract.

In another embodiment, a capsule endoscope configured with a radial scanner produces images that are processed remotely, potentially intercontinentally, by use of the Internet or other wide area networking techology. A receiver located external to the patient forward information of the scanned images through a wireless network to be processed remotely into a full scanned product of digitally overlapped, multiple individual scans as (or after) an endoscopic capsule transits the intestinal tract.

Radial scanning improves distortion issues present in camera systems. Since all the pixels in the scan are equidistant to the photo sensing array and symmetrical along the optical axis, the distortion from the optics is not present. This allows a system design that minimizes optical distortion in the region of the optics used for imaging. Minimal refraction of the light passing through the system also allows for the system to have minimal or no distortion.

The problem of variable resolution is also improved. Since all the pixels in the scans are nearly equidistant to the surface being imaged, the resolution is nearly uniform. Also, since mechanical motion is used to scan down the length of the tube, the resolution is constant and uniform over the length of the tubular structure being scanned.

Since the distance between the radial scanner and the tubular wall being imaged is nearly constant, and the field of view of the scan is limited, the illumination is simple and uniform rather than variable. This also allows the surface area of any one scan to be minimal, which maximizes the optical collection efficiency of the system. This lowers the amount of light and the associated power necessary for illumination and scanning.

The number of useful pixels is also increased in radial scanning. Since the radial scanner is designed in the same geometrical shape as the object being imaged, there is a minimum amount of potentially wasted pixels. The pixels which may be wasted are associated with the requirement in the photo sensor array fabrication for uniform blocks of pixels to be fabricated in a square aligned geometry. This generates a few pixels in each block that will be on the periphery of the collected scans. This is estimated at being a single digit percentage of the number of pixels being used.

Dynamic range is also improved. With the ability to generate a scan scene where the resolution, angle of incidence to the surface, and the illumination are uniform, the dynamic range of the scans collected will be representative of the dynamic range of the reflective properties of the surface material. Whereas camera systems have images whose dynamic range is dominated by illumination and surface angle variations, the radial scanner performance can be optimized for the surface being imaged.

Since the range of the distance between the scanner and the tubular wall being imaged is set by their relative diameters, the required depth of focus is much less for most applications than what is required for camera-based axial imaging systems. This eliminates the need for multiple focus adjustments or the use of high value aperture stops. This results in the scanner being much more optically efficient and able to operate with significantly less illumination power than axial camera systems in the same application.

Radial scanner systems offer an improvement in F-stop performance issues over axial camera systems. Due to the nearly fixed distance between the scanner and the tubular wall being imaged, the optical system can be a low F-number system. This allows a maximization of the operational diameter of the lenses in the system, and maximizes the amount of light that can be handled by the system. As mentioned above, this allows for significant reduction of the required illumination when compared to a bore-aligned camera.

With the ability of the radial scanner to resolve the full circumference of the tube at a common resolution, the need for steerable or restricted field of view systems is eliminated. The radial scanner allows the generation of images of any format at variable resolution and covering any desired amount of the tubular surface.

### CONCLUSION

While specific embodiments of the invention have been described above, it will be appreciated that the invention may be practiced otherwise than as described. For example, the invention may take the form of a computer program containing one or more sequences of machine-readable instructions describing a method as disclosed above, or a data storage medium (e.g., semiconductor memory, magnetic or optical disk) having such a computer program stored therein.

The descriptions above are intended to be illustrative, not limiting. Thus, it will be apparent to one skilled in the art that modifications may be made to the invention as described without departing from the scope of the claims set out below. It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

Embodiments of the present invention have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A radial scanner configured to image the interior of a tube, comprising:
a housing having a transparent window;
a photo-sensing array;
a mirror located within the housing and oriented to direct an image around a circumference of an interior surface of the tube outside the transparent window to the photo-sensing array; and
a light source configured to illuminate the interior surface of the tube,
wherein the photo-sensing array is configured to receive the image around the circumference as a circular line scan.

2. The radial scanner of claim 1, wherein the mirror is a cylindrically symmetrical reflective element or is an element combining reflective and refractive surfaces.

3. The radial scanner of claim 2, wherein the mirror has a hollowed-out conical shape.

4. The radial scanner of claim 1, wherein the mirror is a radial set of prisms oriented such that reflective hypotenuse surfaces of the prisms direct images from the interior surface of the tube to the photo-sensing array.

5. The radial scanner of claim 1, wherein the photo-sensing array comprises a ring of photo-sensing pixels.

6. The radial scanner of claim 1, wherein the housing is an ingestible capsule.

7. The radial scanner of claim 1, wherein the housing is tethered to an external unit configured to be located outside the tube via an optical fiber.

8. The radial scanner of claim 7, wherein the photo-sensing array is located within the external unit.

9. A medical catheter comprising:
a fiber optic cable;
an intra-cavity unit coupled to a distal end of the fiber optic cable; and
an external unit coupled to a proximal end of the fiber optic cable, wherein the external unit comprises:
a photo-sensing array configured to receive a scanned image, and wherein the intra-cavity unit comprises:
a housing coupled to the distal end of the fiber optic cable, the housing having a transparent window;
a mirror located within the housing and oriented to direct an image around a circumference of an interior surface of a tubular structure outside the transparent window to the photo-sensing array via the fiber optic cable.

10. The medical catheter of claim 9, wherein the intra-cavity unit further comprises a lens configured to direct the image from the mirror into the fiber optic cable.

11. The medical catheter of claim 9, wherein the mirror has a conical shape.

12. The medical catheter of claim 9, wherein the mirror is a radial set of prisms oriented such that reflective hypotenuse surfaces of the prisms direct images from the interior surface of the tube to the photo-sensing array.
